(19) United States
(12) Patent Application Publication
(10) Pub. No.: US 2023/0167147 A1
(43) Pub. Date: Jun. 1, 2023

(54) METHOD OF PRODUCING ALIPHATIC GLYCOSIDE COMPOUND OR SUGAR FATTY ACID ESTER COMPOUND

(71) Applicant: TOHOKU UNIVERSITY, Sendai-shi, Miyagi (JP)

(72) Inventors: Kousuke HIROMORI, Sendai-shi, Miyagi (JP); Naoki MAEDA, Sendai-shi, Miyagi (JP); Tomone SASAYAMA, Sendai-shi, Miyagi (JP); Naomi KITAKAWA, Sendai-shi, Miyagi (JP)

(73) Assignee: TOHOKU UNIVERSITY, Sendai-shi, Miyagi (JP)

(21) Appl. No.: 17/927,616

(22) PCT Filed: May 28, 2021
(86) PCT No.: PCT/JP2021/020523

§ 371 Date: Nov. 23, 2022

(30) Foreign Application Priority Data
May 29, 2020 (JP) 2020-094792

(51) Int. Cl. C07H 7/02; C07H 1/00; B01J 39/05

(52) U.S. Cl. CPC C07H 7/02; C07H 1/00; B01J 39/05

(57) ABSTRACT

Provided is a method of producing an aliphatic glycoside compound or a sugar fatty acid ester compound by subjecting an intramolecularly dehydrated sugar and an alcohol or carboxylic acid compound of an aliphatic hydrocarbon to an addition reaction in the presence of an acid catalyst.

### FIELD OF THE INVENTION

The present invention relates to a method of producing an aliphatic glycoside compound or a sugar fatty acid ester compound.

### BACKGROUND OF THE INVENTION

Aliphatic glycoside compounds, such as alkyl glucosides, in which an aliphatic hydrocarbon group is glycosidically bonded to a sugar are suitable as nonionic surfactants derived from natural raw materials having a sugar skeleton as a hydrophilic group. Aliphatic glycoside compounds exhibit higher foamability as compared with general nonionic surfactants. Moreover, since aliphatic glycoside compounds are less irritative to proteins and the skin and are also easily degradable after use, the compounds are highly safe and highly environmental-friendly. For that reason, aliphatic glycoside compounds are used for a wide range of manufactured products such as facial cleansers, shampoos, and dishwashing detergents.

Regarding an industrial synthesis method (production method) for an aliphatic glycoside compound, there is available a direct method of reacting a sugar (for example, glucose) with a higher alcohol in the presence of an acid catalyst, or an indirect method of reacting in advance a sugar with a lower alcohol such as butanol to first synthesize a glycoside and then performing an alcoholysis with a higher alcohol. For both the synthesis methods, the reaction ratio is increased by using a homogeneous phase acid (for example, a Broensted acid such as an inorganic acid or an organic acid) as an acid catalyst and performing the reaction at a high temperature of 100° C. or higher and under reduced pressure while removing water or a lower alcohol, both of which are by-produced.

However, in the synthesis methods of the related art, side reactions such as a polymerization reaction of a sugar also proceed (polysaccharides are by-produced by condensation reactions between sugars), and color deterioration (coloring) such as browning of the reaction product becomes a problem. Moreover, decomposition reactions of sugars also occur. Further, in a direct method, by-products in which the position of formation of the glycoside bond (hydroxy group) is different may be produced, and a decrease in purity of the reaction product also causes a problem. In addition to these problems, in the synthesis methods of the related art, the load of a purification step for removing unreacted substances, and particularly by-products, from the reaction mixture is also large.

Under these circumstances, with regard to by-production of polysaccharides, as a direct method capable of reducing this by-product, a production method for an alkyl glycoside including reacting a sugar with a higher alcohol in the presence of an emulsifier and an acid catalyst, the production method using an aqueous sugar solution as the sugar and using an alkyl glycoside including an alkyl pentoside as an emulsifier, has been proposed in Patent Literature 1.

On the other hand, sugar fatty acid ester compounds in which an aliphatic hydrocarbon group is ester-bonded to a sugar, also have a sugar skeleton as a hydrophilic group and an aliphatic hydrocarbon group as an oleophilic group and are used as nonionic surfactants and the like. Regarding a synthesis method for such a sugar fatty acid ester compound, as is the case of the aliphatic glycoside compounds, there is known a direct method of reacting a sugar and an aliphatic carboxylic acid in the presence of an acid catalyst, or an indirect method of performing a transesterification reaction by using a sugar and a fatty acid ester. In regard to this direct method, the production method described in Patent Literature 2 may be mentioned, although the production method is not a technology for synthesizing a sugar fatty acid ester compound.

### CITATION LIST

### Patent Literatures

Patent Literature 1: JP-A-2014-125474 (“JP-A” means an unexamined published Japanese patent application)
Patent Literature 2: JP-A-2013-159685

### SUMMARY OF THE INVENTION

### Technical Problem

With regard to the production method described in Patent Literature 1, it is described that an alkyl glycoside can be directly synthesized by using a biomass-derived aqueous sugar solution as it is, while reducing by-production of polyglucose. However, with regard to this production method, the amount of by-production of polyglucose can be reduced as compared to the synthesis methods of the related art; however, in reality (Table 1), the amount of by-production can be reduced by up to 5% to 22% by mass only, and there is room for improvement. Further, a purification step is required in order to use the alkyl glycoside as a nonionic surfactant. Moreover, in order to realize prevention of coloration and an increase in purity, the production method described in Patent Literature 2 essentially requires a post-treatment step (purification step) that uses an anion exchange resin, and this production method has a problem that even when a sugar fatty acid ester compound is produced by using a sugar, a post-treatment step still needs to be carried out.

By overcoming the above-described problems, the present invention provides a method capable of highly suppressing the above-described side-reactions such as a condensation reaction between sugars and producing a high-purity aliphatic glycoside compound or sugar fatty acid ester compound with a high conversion ratio by a simple and convenient process.

### Solution to Problem

The present inventors found that in the methods of producing an aliphatic glycoside compound and a sugar fatty acid ester compound, instead of the conventional synthesis method such as a dehydration condensation or an exchange reaction, when an intramolecularly dehydrated sugar is used and brought into contact with an alcohol compound or carboxylic acid compound of an aliphatic hydrocarbon in the presence of an acid catalyst, the cyclic ether structure of the intramolecularly dehydrated sugar and the above-described compound undergo an addition reaction (a glycoside reaction or a ring-opening addition reaction), and an intended aliphatic glycoside compound or sugar fatty acid ester compound can be directly synthesized at a high conversion ratio. Furthermore, with regard to these addition reactions, the present inventors found that the occurrence of side reactions such as a decomposition reaction or a condensation reaction of the intramolecularly dehydrated sugar, more particularly a reaction that uses hydroxy groups of the by-products as reaction sites, can be effectively suppressed, and a high-purity target compound in which coloration and contamination of impurities are suppressed can be produced by a simple and convenient process. The present inventors repeated more studies based on these findings and finally completed the present invention.

That is, the object of the present invention was achieved by the following means.

<1> A method of producing an aliphatic glycoside compound or a sugar fatty acid ester compound, which contains a step of subjecting an intramolecularly dehydrated sugar and an alcohol or carboxylic acid compound of an aliphatic hydrocarbon to an addition reaction in the presence of an acid catalyst.
<2> The method of producing an aliphatic glycoside compound or a sugar fatty acid ester compound described in <1>, wherein the acid catalyst is a solid acid catalyst.
<3> The method of producing an aliphatic glycoside compound or a sugar fatty acid ester compound described in <2>, wherein the solid acid catalyst is a cation exchanger.
<4> The method of producing an aliphatic glycoside compound or a sugar fatty acid ester compound described in any one of <1> to <3>, wherein the intramolecularly dehydrated sugar is an intramolecular dehydration reaction product in which water molecules are eliminated from two hydroxy groups including a hydroxy group bonded to a carbon atom at the 1-position in the cyclic structure.
<5> The method of producing an aliphatic glycoside compound or a sugar fatty acid ester compound described in any one of <1> to <4>, wherein the intramolecularly dehydrated sugar is an intramolecularly dehydrated sugar of an aldose.
<6> The method of producing an aliphatic glycoside compound or a sugar fatty acid ester compound described in any one of <1> to <5>, wherein the intramolecularly dehydrated sugar is levoglucosan.
<7> The method of producing an aliphatic glycoside compound or a sugar fatty acid ester compound described in any one of <1> to <6>, wherein the number of carbon atoms constituting the alcohol or carboxylic acid compound is 1 to 22.
<8> The method of producing an aliphatic glycoside compound or a sugar fatty acid ester compound described in any one of <1> to <7>, wherein the aliphatic hydrocarbon is a saturated aliphatic hydrocarbon.
<9> The method of producing an aliphatic glycoside compound or a sugar fatty acid ester compound described in any one of <1> to <8>, wherein a mixture of the alcohol or carboxylic acid compound of an aliphatic hydrocarbon and the intramolecularly dehydrated sugar is brought into contact with the acid catalyst.
<10> The method of producing an aliphatic glycoside compound or a sugar fatty acid ester compound described in <9>, wherein the mixture is brought into contact with the acid catalyst by a batch method or a continuous method.
<11> The method of producing an aliphatic glycoside compound or a sugar fatty acid ester compound described in <9> or <10>, wherein the mixture is a mixed liquid obtained by dissolving at least a portion of the intramolecularly dehydrated sugar in the alcohol or carboxylic acid compound of an aliphatic hydrocarbon.

A numerical value range indicated by using the term “to” in the present specification means a range including the numerical values described before and after the term “to” as the lower limit value and the upper limit value, respectively.

### Advantageous Effects of Invention

The method of producing an aliphatic glycoside compound or a sugar fatty acid ester compound of the present invention suppresses the above-described side reactions such as a condensation reaction between sugars, and a high-purity aliphatic glycoside compound or sugar fatty acid ester compound can be produced at a high conversion ratio of an intramolecularly dehydrated sugar by a relatively simple and convenient process.

The above-described and other features and advantages of the present invention will appear more fully from the following description.

### DESCRIPTION OF EMBODIMENTS

The method of producing an aliphatic glycoside compound and the method of producing a sugar fatty acid ester compound of the present invention (hereinafter, may be simply referred to as production methods of the present invention) include a method of producing an aliphatic glycoside compound (hereinafter, may be simply referred to as a glycoside production method of the present invention) and a method of producing a sugar fatty acid ester compound (hereinafter, may be simply referred to as a sugar ester production method of the present invention), by means of a compound (reactant) that is subjected to an addition reaction with an intramolecularly dehydrated sugar.

As an example of the reaction scheme according to the production method of the present invention, a reaction scheme of each production method of using levoglucosan (LG) as an intramolecularly dehydrated sugar is shown below. In Scheme 1, R¹ designates an aliphatic hydrocarbon group, and in Scheme 2, R² designates a hydrogen atom or an aliphatic hydrocarbon.

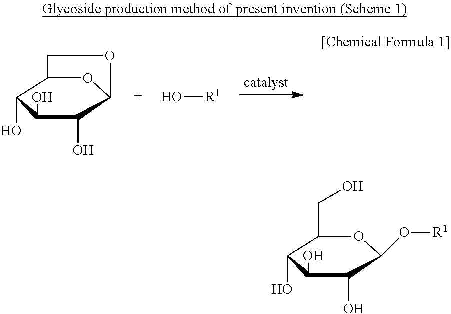

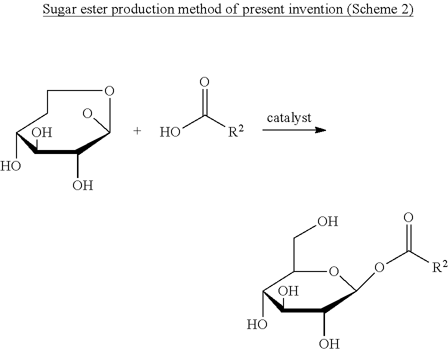

First, raw material compounds that are used for the production methods of the present invention will be described.

### (Intramolecularly Dehydrated Sugar)

The intramolecularly dehydrated sugar used for the production methods of the present invention refers to a sugar that is intramolecularly dehydrated (also referred to as anhydrosugar).

As the sugar from which the intramolecularly dehydrated sugar is derived, various sugars can be used without any particular limitation, and the sugar may be any of a monosaccharide, an oligosaccharide, a polysaccharide, and the like. As described above, the present invention can effectively suppress the occurrence of side reactions such as a decomposition reaction and a condensation reaction of sugars, and a reaction that uses a hydroxy group of a by-product as a reaction site. Therefore, the present invention may use not only a ketose but also an aldose, which is conventionally likely to cause a decrease in purity.

The monosaccharide is not particularly limited, and examples thereof include aldopentoses such as ribose, arabinose, xylose, and lyxose; and aldohexoses such as allose, altrose, glucose, mannose, gulose, idose, galactose, and talose. The oligosaccharide is not particularly limited, and examples thereof include disaccharides such as maltose, cellobiose, lactose, and sucrose; and trisaccharides such as maltotriose. The polysaccharide is not particularly limited, and examples thereof include hemicellulose, inulin, dextrin, dextran, xylan, starch, and hydrolyzed starch.

The sugar from which the intramolecularly dehydrated sugar is derived is preferably a monosaccharide from the viewpoint of reactivity, and above all, a pentose or a hexose is preferred, a hexose is more preferred, while in a case in which the product is used as a suitable nonionic surfactant or the like, glucose is even more preferred. As the sugar from which the intramolecularly dehydrated sugar is derived, it is preferable to use an aldose from the viewpoint that the features of the present invention can be utilized (the operating effect is effectively realized).

The intramolecularly dehydrated sugar may be a compound in which water molecules are intramolecularly eliminated from the above-described sugar, and the number of water molecules to be eliminated from one molecule of sugar is not particularly limited but is usually one molecule.

The water molecules to be dehydrated from one molecule of sugar may be eliminated from any hydroxy group among the hydroxy groups carried by the sugar and are appropriately selected. In the present invention, from the viewpoint of reactivity, and further from the viewpoint that a high-purity target compound can be produced by effectively suppressing the occurrence of the above-described side reactions that cannot be suppressed in the production methods of the related art, the intramolecularly dehydrated sugar is preferably an intramolecular dehydration reaction product in which water molecules are eliminated from two hydroxy groups including a hydroxy group bonded to the carbon atom at the 1-position in the cyclic structure of the sugar (1,6-anhydrosugar: n designates the position of the carbon atom to which a hydroxy group is bonded in the cyclic structure of the sugar, and for example, n is 2, 3, 4, or 6). In this case, the other hydroxy group is not particularly limited; however, from the viewpoint of reactivity, the other hydroxy group is preferably a hydroxy group that is bonded to the carbon atom at the 6-position in the cyclic structure of the sugar (1,6-anhydrosugar).

The intramolecularly dehydrated sugar is preferably an intramolecularly dehydrated sugar of an aldose, and (α- or β-)levoglucosan, which is a 1,6-anhydrosugar of (α- or β-)glucose, is particularly preferred.

The intramolecularly dehydrated sugar may include water of crystallization, water of hydration, and the like.

The intramolecularly dehydrated sugar may be appropriately synthesized, or a commercially available product may be used. For example, an intramolecularly dehydrated sugar that is by-produced in a large quantity in a thermal decomposition reaction of cellulose, which is a non-edible biomass, can be used.

In the production method of the present invention, the intramolecularly dehydrated sugar can be used as a solution or a dispersion liquid; however, it is preferable that the intramolecularly dehydrated sugar is used as a compound (usually in a solid state).

### (Alcohol Compound and Carboxylic Acid Compound)

The alcohol compound used for the production method of the present invention is an (aliphatic) alcohol compound in which at least one hydrogen atom of an aliphatic hydrocarbon is substituted with a hydroxy group, and the carboxylic acid compound is a carboxylic acid compound of an aliphatic hydrocarbon (formic acid or an (aliphatic) carboxylic acid compound in which at least one hydrogen atom of an aliphatic hydrocarbon is substituted with a carboxy group).

The aliphatic hydrocarbon is appropriately determined according to the use application and the like of the intended aliphatic glycoside compound and sugar fatty acid ester compound. This aliphatic hydrocarbon may be a saturated aliphatic hydrocarbon or may be an unsaturated aliphatic hydrocarbon; however, a saturated aliphatic hydrocarbon is preferred. The carbon chain structure of the fatty acid hydrocarbon is not particularly limited and may be any of a straight chain, a branched chain, and a cyclic chain; however, from the viewpoint of reactivity, a straight chain or a branched chain is preferred, and a straight chain is more preferred.

The number of carbon atoms constituting the alcohol compound and the carboxylic acid compound (the number of carbon atoms forming a carbon chain of an aliphatic hydrocarbon constituting the alcohol compound, and the total number of the number of carbon atoms forming a carbon chain of an aliphatic hydrocarbon constituting the carboxylic acid compound and the carbonyl carbon atom of the ester bond) is appropriately determined in consideration of reactivity and further the usefulness (use application) of the intended compound and is not particularly limited. The number of carbon atoms is, for example, from the viewpoint of reactivity, preferably 1 to 30, more preferably 1 to 22, and further preferably 1 to 12, and from the viewpoint of usefulness of the intended compound (particularly as a nonionic surfactant or the like) in addition to reactivity, the number of carbon atoms is further preferably 6 to 22, and particularly preferably 8 to 16.

With regard to the alcohol compound and the carboxylic acid compound, the degree of substitution of the hydroxy group or carboxy group is not particularly limited; however, from the viewpoint of reactivity, the hydroxy group or carboxy group is preferably primary. The number of hydroxy groups or carboxy groups carried by one molecule of the alcohol compound or carboxylic acid compound is not particularly limited and can be set to 1 to 10; however, the number of hydroxy groups or carboxy groups is usually one (monoalcohol compound or monocarboxylic acid compound).

### (Acid Catalyst)

As the acid catalyst to be used for the present invention, a known acid catalyst can be used without particular limitation as long as it is an acid catalyst that is ordinarily used for a glycoside reaction or a ring-opening addition reaction. Examples include a Broensted acid (homogeneous phase acid catalyst) and a solid acid catalyst (heterogeneous phase solid acid catalyst). As the acid catalyst, one kind or two or more kinds thereof can be used.

### —Broensted Acid—

The Broensted acid is not particularly limited; however, examples thereof include organic acids such as para-toluenesulfonic acid and methanesulfonic acid; and inorganic acids such as sulfuric acid, hydrochloric acid, nitric acid, and phosphoric acid.

### —Solid Acid Catalyst—

The solid acid catalyst is, for example, a solid compound having an active site (acid site) and exhibits a catalytic action, and examples of the active site carried by the solid acid catalyst include a Broensted acid site and a Lewis acid site, which solid acid catalysts usually have. Examples of such a solid acid catalyst include a silica-alumina catalyst, a zeolite catalyst, and a cation exchanger (cation exchange resin), and a cation exchanger is preferred.

As the cation exchanger, a strongly acidic cation exchange resin, a weakly acidic cation exchange resin, and the like can be used without particular limitation, and among them, from the viewpoint of the reaction rate, a strongly acidic cation exchange resin is preferred. The cation exchanger may be any of a porous type (porous), a highly porous type (porous), and a gel type; however, it is preferable that the cation exchanger is a porous type from the viewpoint of reactivity. Here, the gel type is a cation exchanger formed of a crosslinked polymer having a uniform (particle) interior. The porous type has a structure in which physical holes (pores) are formed in a gel type cation exchanger. The highly porous type is a cation exchanger having a high degree of crosslinking and having a structure with a larger specific surface area and a larger pore volume than a porous type.

Regarding the cation exchanger, those in which the resin skeleton as an insoluble carrier has various chemical structures can be used. Examples of the resin that constitutes an insoluble carrier include synthetic polymers such as a polystyrene and a polyacrylic acid which are crosslinked with divinylbenzene or the like, a crosslinked poly(meth)acrylic acid ester, and a phenol resin; and naturally produced crosslinked bodies of polysaccharides, such as cellulose. Above all, a synthetic polymer is preferred, and a crosslinked polystyrene is more preferred. The degree (extent) of crosslinking is dependent on the amount of use of divinylbenzene with respect to the total amount of monomers constituting the resin, and for example, the degree of crosslinking is selected in the range of 1% to 30% by mass. At that time, as the degree of crosslinking is lower, a compound having a larger molecular size is easily diffused in the inner part of the resin; however, since the concentration of the active site becomes smaller, there is an optimum value so that the addition reaction exhibits high catalytic activity.

The active site (ionic functional group) of the cation exchanger is not particularly limited; however, examples include a sulfonic acid group and a carboxy group.

Examples of the cation exchanger include DIAION (registered trademark) PK series, DIAION (registered trademark) SK series, RCP160M (all manufactured by Mitsubishi Chemical Corporation), Amberlite series, and Amberlyst series (all manufactured by The Dow Chemical Company). These cation exchangers have a skeleton of a polymer of styrene and divinylbenzene and have a sulfonic acid group as an ionic functional group (exchange group). Among the above-described DIAION (registered trademark) PK series, PK208LH, PK212LH, and PK216LH are of the porous type, SK104H among the DIAION (registered trademark) SK series is of the gel type, and RCP160M is of the highly porous type.

The cation exchanger is usually used in the H⁺ form (free acid form) exhibiting catalytic activity. Moreover, the above-described commercially available cation exchangers are in the H⁺ form (≥99 mol %) in which the ionic functional groups all exhibit a catalytic activity at the time of factory shipment; however, since the cation exchangers are in a water-swollen state, it is preferable to perform a treatment of bringing a cation exchanger into a state of being swollen in a reactant (alcohol-swollen state or carboxylic acid-swollen state) as a pretreatment as appropriate. This pretreatment can be carried out by an ordinarily used method under ordinarily used conditions. For example, the method described in Fuel., 139, 11-17 (2015) can be applied.

Regarding the shape of the solid acid catalyst (cation exchanger), any shape such as a membrane shape and a particulate shape can be selected according to the form of use thereof; however, a particulate shape is preferred. Moreover, in the case of a particulate shape, the particle diameter thereof is not particularly limited and is usually 10 μm or more, preferably 100 μm or more, and more preferably 200 μm or more. The upper limit of the particle diameter is usually 2 mm or less, preferably 1.5 mm or less, and more preferably 1 mm or less. When the particle diameter is too small, handling may be difficult, and when the particle diameter is too large, the reaction rate may be decreased. The particle diameter of the solid acid catalyst can be measured by means of an optical microscope.

The solid acid catalyst (cation exchanger) can be reused after being used in the addition reaction as needed, by subjecting the solid acid catalyst to a regeneration treatment. For the regeneration treatment, an ordinarily used method can be applied, and for example, a treatment of substituting (swelling) with an alcohol compound or the like, which is a reactant, similarly to the above-mentioned pretreatment may be mentioned. With regard to the regeneration treatment, a treatment of separating or collecting the solid acid catalyst after the addition reaction by a solid-liquid separation method such as suction and filtration or the like and a treatment of washing the solid acid catalyst may be carried out as appropriate. By this regeneration treatment, the reactant and reaction product remaining in the inside or on the surface of the solid acid catalyst can be separated.

### (Solvent)

In the production method of the present invention, a solvent that dissolves the intramolecularly dehydrated sugar may be used, while either the alcohol compound or the carboxylic acid compound can be used in excess (as long as it is liquid under the reaction conditions) with respect to the molecularly dehydrated sugar, and these compounds can be used as a reactant as well as a solvent. As a result, enhancement of reactivity and simplification of the production process can be achieved. For that reason, it is preferable that in the production method of the present invention, particularly in the glycoside production method of the present invention, a solvent other than the alcohol compound or the carboxylic acid compound (also referred to as reactant) is not used (addition reaction in the absence of a solvent). According to the present invention, the absence of a solvent means to include an embodiment that does not use a solvent other than the alcohol compound or the carboxylic acid compound, as well as an embodiment of using (containing) the above-described solvents to the extent that does not inhibit the addition reaction (for example, 10% by mass or less with respect to the sum of the reactants and the solvent).

The solvent that may be used for the production method of the present invention is not particularly limited as long as the solvent does not inhibit the addition reaction, and various solvents may be mentioned.

### (Other Components)

In the production method of the present invention, components other than the intramolecularly dehydrated sugar, the alcohol compound or carboxylic acid compound, and the acid catalyst can also be used. For example, an emulsifier that promotes or assists the dissolution of the intramolecularly dehydrated sugar may be mentioned. The emulsifier is not particularly limited; however, from the viewpoint that it is not necessary to remove the emulsifier that has been used, it is preferable that the emulsifier is a compound of the same kind as the intended compound (aliphatic glycoside compound or sugar fatty acid ester compound). In this case, the emulsifier also accomplishes a function of promoting the addition reaction.

Next, the production steps of the present invention will be described.

In the production method of the present invention, an intramolecularly dehydrated sugar and an alcohol compound or a carboxylic acid compound are subjected to an addition reaction by bringing the compounds into contact with each other in the presence of an acid catalyst.

The amount of use of the alcohol compound (at the time of initiation of the reaction) in this step is usually set to be equal to or more than the stoichiometric amount with respect to 1 mol of the intramolecularly dehydrated sugar. The upper limit of the amount of use is not particularly limited, and when considering productivity, economic efficiency, and the like, for example, the upper limit is preferably 1,000 molar times or less, and more preferably 100 molar times or less, with respect to 1 mol of the intramolecularly dehydrated sugar.

According to the present invention, since the reactivity can be enhanced by using an alcohol compound as a solvent for the intramolecularly dehydrated sugar, it is preferable to use the alcohol compound in an excess amount with respect to the intramolecularly dehydrated sugar, without using a solvent other than the alcohol compound. The amount of use of the alcohol compound at this time is set to an appropriate amount, in consideration of the reactivity (reaction rate) of the intramolecularly dehydrated sugar, which is a solid at ordinary temperature and normal pressure (25° C., 101 kPa), practically the amount of dissolution of the intramolecularly dehydrated sugar in the alcohol compound. For example, the amount of use (excess amount) of the alcohol compound can be set to an amount of use in which the mixture of the intramolecularly dehydrated sugar and the alcohol compound has a homogeneous phase under the reaction conditions. More specifically, it is preferable that the amount of use is set to an amount in which a portion or the entirety of the intramolecularly dehydrated sugar is dissolved in the alcohol compound. Incidentally, in a case where the addition reaction is carried out by a batch method that will be described below, the amount of use is set in further consideration of the immersion state of the acid catalyst. Here, a portion of the intramolecularly dehydrated sugar refers to the extent to which the remaining portion of the intramolecularly dehydrated sugar present without being dissolved in the alcohol compound is dissolved (by an emulsification action of the reaction product) in the alcohol compound until the completion of the addition reaction (in a continuous method that will be described below, passing through the acid catalyst), and the portion is appropriately set in consideration of the solubility of the intramolecularly dehydrated sugar, the reaction temperature, the reaction time, the size of the reaction vessel, and the like. The amount of use in the case of using the alcohol compound in excess is not particularly limited, and an example thereof may be the same range as that of the above-described amount of use (at the time of initiation of the reaction).

According to the present invention, in a case where a solid acid catalyst is used as the acid catalyst, the amount of use of the alcohol compound is set to an amount that does not include the amount of use of the alcohol compound used for alcohol swelling of the solid acid catalyst.

The amount of use of the carboxylic acid compound (at the time of initiation of the reaction) is usually set to be similar to the above-mentioned amount of use of the alcohol compound.

The amount of use of the acid catalyst (at the time of initiation of the reaction) is set to be equal to or more than the amount in which the active sites in the acid catalyst function as a catalyst for the addition reaction between the intramolecularly dehydrated sugar and the alcohol compound or the carboxylic acid compound.

The amount of use of the acid catalyst itself may be any amount in which the active sites in the acid catalyst are present in a catalytic amount, and for example, the amount of use is appropriately set in consideration of the quantity of active sites of the acid catalyst, the reaction time (time for passage in the continuous method that will be described below), further the immersion state in the batch method, and the like. More specifically, in a case where the addition reaction is performed by the batch method that will be described below by using a solid acid catalyst, the amount of use of the acid catalyst can be set to, for example, 5% to 60% by mass with respect to the total amount of the intramolecularly dehydrated sugar and the alcohol compound or the carboxylic acid compound. On the other hand, in a case where the addition reaction is performed by the continuous method that will be described by using a solid acid catalyst, the amount of use can be set to, for example, 10 to 250 mL/min as the amount of liquid flow of the raw material mixture (mixture of the intramolecularly dehydrated sugar and the alcohol compound or the carboxylic acid compound) per 1 L of the solid acid catalyst.

In the production method of the present invention, an intramolecularly dehydrated sugar and an alcohol compound or a carboxylic acid compound are preferably set to the above-described amounts of use and then are subjected to an addition reaction in the presence of an acid catalyst. The addition reaction can be carried out by bringing the intramolecularly dehydrated sugar, the alcohol compound or the carboxylic acid compound, and the acid catalyst into contact with each other (mixing, flowing through, or the like). The method of bringing into contact is as will be described below, and the order of bringing into contact is not particularly limited. Since the intramolecularly dehydrated sugar is usually not easily dissolved in the alcohol compound or the carboxylic acid compound, it is preferable to mix the intramolecularly dehydrated sugar and the alcohol compound or the carboxylic acid compound in advance to prepare a (preliminary) mixture and to bring this (preliminary) mixture into contact with the acid catalyst. From the viewpoint of rapidly conducting the addition reaction, the (preliminary) mixture may be a solution in which the intramolecularly dehydrated sugar is dissolved in the alcohol compound or the like; however, in the present invention, the (preliminary) mixture is not necessarily a solution and may be a mixed liquid (dispersion liquid) in which at least a portion of the intramolecularly dehydrated sugar is dissolved in the alcohol compound or the carboxylic acid compound. Here, the term portion of the intramolecularly dehydrated sugar is as described above.

It is preferable that the intramolecularly dehydrated sugar, the alcohol compound or the carboxylic acid compound, and the acid catalyst are set to the following reaction temperature in a state of being in contact with each other; however, depending on the method of contacting, for example, each of the (preliminary) mixture and/or the acid catalyst can be preheated to the reaction temperature and brought into contact with each other.

The (preliminary) mixture can be prepared by mixing the intramolecularly dehydrated sugar and the alcohol compound or the like by an ordinarily used method. The preparation conditions can be appropriately determined in consideration of the solubility of the intramolecularly dehydrated sugar and the like, and for example, the heating conditions may be mentioned. The mixing temperature at this time is not particularly limited; however, for example, the same temperature range as the reaction temperature described below may be mentioned.

The addition reaction conditions for both the reactants are not particularly limited as long as the conditions are conditions in which the addition reaction proceeds, and the reaction temperature, the reaction time, the reaction atmosphere, the reaction pressure, the form of contact, the contacting conditions, and the like are appropriately set.

The reaction temperature at the time of the addition reaction is not particularly limited, and for example, the reaction temperature can be set to be equal to or higher than room temperature (25° C.). From the viewpoints of the reaction rate and the solubility of the intramolecularly dehydrated sugar in the alcohol compound or the like, the reaction temperature is preferably 30° C. to 150° C., more preferably 40° C. to 80° C., and further preferably 50° C. to 70° C.

The above-described reaction temperature refers to the temperature in a state in which the intramolecularly dehydrated sugar, the alcohol compound or the carboxylic acid compound, and the acid catalyst are in contact with each other; however, it is preferable that the acid catalyst, particularly the solid acid catalyst, is also at the above-described reaction temperature.

The reaction time is appropriately set based on other reaction conditions such as the reaction temperature, the amount of use of the acid catalyst, and further the conversion ratio of the intramolecularly dehydrated sugar and the like, and for example, the reaction time can be set to be 10 minutes or longer and 12 hours or shorter. Incidentally, in the continuous method, the intramolecularly dehydrated sugar and the alcohol compound or the carboxylic acid compound are transferred into an acid catalyst (usually, a solid acid catalyst) within the above-described reaction time. The transfer time at this time can be set to, for example, about 0.1 to 100 mL/minute per 1 L of the acid catalyst.

The reaction atmosphere is appropriately set according to the contacting method, and for example, the reaction atmosphere can be set to an inert gas atmosphere of nitrogen gas, argon gas, or the like, in addition to the air atmosphere.

Regarding the reaction pressure conditions, pressurized conditions or reduced pressure conditions can be appropriately selected; however, from the viewpoint of suppressing evaporation of the alcohol compound or the like, atmospheric pressure conditions or pressurized conditions can be employed, and for example, the reaction pressure conditions can be set to 50 to 500 kPa.

In the production method of the present invention, the contact between the reactants and the acid catalyst can be carried out by a batch method or by a continuous method (flow method). The batch method is a method of briefly performing charging of reactants and the like and acquisition of a reaction product, and for example, the batch method refers to an operation of charging a one-time production portion of the reactants and the acid catalyst into a reaction apparatus such as a glass reactor, a shaker, or a constant-temperature tank, performing an addition reaction, taking out the obtained reaction product, and then performing the next addition reaction in the same manner. Regarding the contacting method for the batch method, an ordinarily employed method can be applied without particular limitation, and examples include a stirring method and a shaking method. The stirring conditions and the shaking conditions are appropriately set, and for example, the conditions employed in the Examples may be mentioned as an example of the shaking conditions. The continuous method is a method of continuously performing charging of reactants and the like and acquisition of a reaction product, and for example, the continuous method refers to an operation of continuously supplying reactants and an acid catalyst to a reaction apparatus (preferably, reactants are continuously supplied to a reaction apparatus packed with an acid catalyst (usually, a solid acid catalyst)) and at the same time, continuously taking out the obtained reaction product. Regarding the contacting conditions for the continuous method, an ordinarily employed method can be applied without particular limitation, and examples include a method of transferring (causing to flow through the inside of the catalyst layer) reactants (usually, a mixture of reactants) into a packed bed (catalyst layer) packed with an acid catalyst in a circulated system or a counter-current system, and a fluidized bed reaction method.

In the production method of the present invention, the alcohol compound or the carboxylic acid compound is subjected to an addition reaction to a cyclic ether structure formed by intramolecular dehydration of the intramolecularly dehydrated sugar through the above-described contacting.

Since the reaction site of the addition reaction is specific and selective in this manner, the intended addition reaction can be caused with a high selectivity ratio without causing a polymerization reaction or a decomposition reaction of a sugar as is the case of the synthesis methods of the related art of using a sugar. Moreover, production of by-products in which the position of formation of a glycoside bond or an ether bond is different can also be highly suppressed. Therefore, the addition reaction step can also be conveniently carried out. Further, when a solid acid catalyst is used as the acid catalyst, the solid acid catalyst can be conveniently separated and removed from the reaction mixture as will be described below. Accordingly, a high-purity target compound can be produced by a convenient production process as the production method of the present invention.

In addition, as the intramolecularly dehydrated sugar for the production method of the present invention, an intramolecularly dehydrated sugar that is by-produced in a large amount in a thermal decomposition reaction of cellulose, which is a non-edible biomass, can be used. Conventionally, when an aliphatic glycoside compound or a sugar fatty acid ester compound is produced using the intramolecularly dehydrated sugar, production is achieved by a multi-stage reaction of adding water to the intramolecularly dehydrated sugar to convert the intramolecularly dehydrated sugar to a sugar, and then performing a glycoside reaction or an esterification reaction. However, in the production method of the present invention, the intramolecularly dehydrated sugar as a by-product of thermal decomposition of a non-edible biomass can be supplied to an addition reaction as it is (without being converted to a sugar), and in this case, the intramolecularly dehydrated sugar can be effectively utilized.

A reaction mixture can be obtained by bringing the reactants and the acid catalyst as described above. An intended aliphatic glycoside compound or sugar fatty acid ester compound can be obtained from the obtained reaction mixture by appropriately applying an ordinarily used method. For example, in a batch method of using a solid acid catalyst, the reaction mixture is separated from the solid acid catalyst by an ordinarily used solid-liquid separation method, and in a continuous method, an effluent that has passed a packed bed of the solid acid catalyst is collected to obtain a reaction mixture from which the solid acid catalyst has been removed. Next, an intended aliphatic glycoside compound or sugar fatty acid ester compound can be obtained by removing the alcohol compound or carboxylic acid compound by evaporation or the like. On the other hand, in a method of using a homogeneous phase acid catalyst, an intended aliphatic glycoside compound or sugar fatty acid ester compound can be obtained by, for example, carrying out a homogeneous phase acid catalyst removal step and a purification step as appropriate by ordinarily used methods, and then removing the alcohol compound or the carboxylic acid compound.

According to the present invention, when a porous type or highly porous type solid acid catalyst is used as the solid acid catalyst (cation exchanger), the reaction product may remain inside the cation exchanger. Therefore, it is preferable to elute (collect) the reaction product from the cation exchanger after reaction. The method of eluting the reaction product is not particularly limited; however, the above-described regeneration treatment can be applied, and specifically, a method of bringing the cation exchanger into contact (immersion) with a solvent such as an alcohol compound, or causing a solvent such as an alcohol compound to flow through the cation exchanger may be mentioned.

### (Aliphatic Glycoside Compound and Sugar Fatty Acid Ester Compound)

The aliphatic glycoside compound and the sugar fatty acid ester compound, which are obtained by the production method of the present invention, are compounds in which an aliphatic hydrocarbon group is introduced into any one of two hydroxy groups that cause a dehydration reaction in a sugar (cyclic structure) from which the intramolecularly dehydrated sugar used as a reactant is derived, by means of a glycoside bond or an ester bond (—CO—O—: an oxygen atom is bonded to the sugar (cyclic structure)).

When levoglucosan (LG) is used as the intramolecularly dehydrated sugar, the specific chemical structures of the aliphatic glycoside compound and the sugar fatty acid ester compound are as shown in the above-described scheme 1 and scheme 2, and the aliphatic glycoside compound and the sugar fatty acid ester compound are compounds in which an aliphatic hydrocarbon group or the like (R¹ or R²) is introduced into the carbon atom at the 1-position of the sugar (cyclic structure) by means of a glycoside bond or an ester bond.

The aliphatic glycoside compound and the sugar fatty acid ester compound have a variety of use applications as described above, and for example, the compounds are suitable as nonionic surfactants. Therefore, the production method of the present invention can produce a compound having a wide variety of use applications, particularly a compound suitable as a nonionic surfactant, with high purity and high yield.

The production method of the present invention can produce (synthesize) an aliphatic glycoside compound or a sugar fatty acid ester compound by highly suppressing a condensation reaction (side reaction) between sugars. The aliphatic glycoside compound or sugar fatty acid ester compound obtainable by the production method of the present invention is such that coloration caused by a condensation reaction between sugars occurs to a lower extent, and contamination of impurities such as by-products is also highly suppressed so that the compound exhibits high purity.

Moreover, the production method of the present invention can produce the above-mentioned high-purity compound at a high conversion ratio of the intramolecularly dehydrated sugar and by a convenient production process (addition reaction, and in a suitable embodiment, a separation step of the acid catalyst and a purification step of the target compound).

### EXAMPLES

Hereinafter, the present invention will be described in more detail based on Examples; however, the technical scope of the present invention is not intended to be limited in any way by these descriptions. Incidentally, unless particularly stated otherwise in the following Examples, general methods known to those ordinarily skilled in the art were followed.

In the following Examples, levoglucosan, which is a 1,6-anhydrosugar, was used as the intramolecularly dehydrated sugar. Levoglucosan was a product manufactured by Fujifilm Wako Chemicals Corporation and had a purity of higher than 97%.

In the following Examples, PK208LH (trade name, manufactured by Mitsubishi Chemical Corporation) was used as a cation exchanger, which is a solid acid catalyst. The cation exchanger was caused to swell with an alcohol compound that was used as a reactant, before use. Swelling of the cation exchanger was performed by circulating the alcohol compound through a packed bed of the cation exchanger by an ordinarily used method.

### Example 1

Reactants (ethanol as the alcohol compound, and the levoglucosan as the intramolecularly dehydrated sugar) and a cation exchanger were brought into contact with each other by a batch method without using a solvent other than ethanol (in the absence of a solvent), which was a reactant, to produce ethyl glycoside.

Specifically, ethanol and levoglucosan were mixed at 60° C. to prepare a preliminary mixture having a levoglucosan concentration of 0.30 mol/L. This preliminary mixture was an ethanol solution of levoglucosan.

Next, 60 g of the obtained preliminary mixture (60° C.) was introduced into a glass reactor, and a cation exchanger (ethanol-swollen product of the above-described PK208LH) preheated to 60° C. was introduced into the glass reactor to a concentration of 33% by mass of the total reaction system. This glass reactor (reaction mixture and cation exchanger) was shaken for 6 hours under the atmospheric pressure conditions and under the conditions of a shaking width of 50 mm and a shaking rate of 150 spm to perform an addition reaction.

During the addition reaction, a small amount of the reaction liquid was collected at predetermined time intervals, the reaction liquid was diluted with ethanol, the conversion ratio of levoglucosan was traced and determined by using an HPLC (Waters Corp., Milford, Mass., USA) system under the following conditions. As a result, the conversion ratio of levoglucosan calculated by the following Formula 1 became 100% after 6 hours.

### (HPLC Measurement Conditions)

The measurement of HPLC can be carried out by appropriately applying a known method that is used for an analysis of a general sugar or sugar fatty acid ester. For example, measurement can be made by using a reversed phase column (ODS column or the like) or an NH₂ column was used as a column and an RI (differential refractive index meter) or an ELS (evaporation light scattering system) as a detector and using a water/acetonitrile mixed solution as an eluent. In the present Example, an NH₂ column was used as a column and an ELS was used as a detector, respectively.

### (Conversion Ratio Calculation Formula for Levoglucosan)

Conversion ratio (%)={(C_{LG,0}−C_{LG,t})/C_{LG,0}}×100 (Formula 1)

In Formula 1, C_{LG,0} designates the levoglucosan concentration (charging concentration) used for the addition reaction, and

C_{LG,t} designates the (unreacted) levoglucosan concentration in the reaction liquid after a lapse of the reaction time t.

### Example 2

n-Butyl glycoside was produced in the same manner as in Example 1, except that n-butanol was used instead of ethanol used in Example 1 as the alcohol compound, and the addition reaction time was set to 3 hours. The prepared preliminary mixture had a slight amount of levoglucosan undissolved in n-butanol; however, after completion of the addition reaction, undissolved levoglucosan was not recognized, and the preliminary mixture was obtained as a reaction liquid.

In Example 2, the conversion ratio of levoglucosan became 100% after 3 hours.

### Example 3

n-Hexyl glycoside was prepared in the same manner as in Example 1, except that n-hexanol was used instead of ethanol used in Example 1 as the alcohol compound, and the addition reaction time was set to 2 hours. The prepared preliminary mixture was a dispersion liquid in which a portion of levoglucosan was dissolved in n-hexanol; however, after completion of the addition reaction, undissolved levoglucosan was not recognized, and the preliminary mixture was obtained as a reaction liquid.

In Example 3, the conversion ratio of levoglucosan became 100% after 2 hours.

### Example 4

n-Octyl glycoside was produced in the same manner as in Example 1, except that n-octanol was used instead of ethanol used in Example 1 as the alcohol compound, and the addition reaction time was set to 3 hours. The prepared preliminary mixture was a dispersion liquid in which a portion of levoglucosan was dissolved in n-octanol; however, after completion of the addition reaction, undissolved levoglucosan was not recognized, and the preliminary mixture was obtained as a reaction liquid.

In Example 4, the conversion ratio of levoglucosan became 100% after 3 hours.

The conditions and results of Examples 1 to 4 are summarized below.

TABLE 1

| Example | Alcohol compound | Solvent | Conversion ratio (%) |
|---|---|---|---|
| 1 | Ethanol | None | ~100 |
| 2 | n-Butanol | None | ~100 |
| 3 | n-Hexanol | None | ~100 |
| 4 | n-Octanol | None | ~100 |

As is obvious from the results shown in Table 1, in the method of producing an aliphatic glycoside compound, due to a characteristic synthesis reaction in which instead of a sugar that has been conventionally used as a starting raw material, an intramolecularly dehydrated sugar whose utilization has not been hitherto focused is used to perform an addition reaction with an alcohol compound in the presence of a cation exchanger, in all of Examples 1 to 4, even when the type of the alcohol compound and the reaction conditions are changed, the occurrence of the above-described side reactions can be effectively suppressed at a conversion ratio of the intramolecularly dehydrated sugar of almost 100%, and the addition reaction can be accomplished.

As described above, in Examples 1 to 4, since the occurrence of the above-described side reactions in the absence of a solvent is effectively suppressed, and the reaction proceeds at a conversion ratio of about 100%, the reaction liquid obtained in each Example becomes a mixture of an aliphatic glycoside compound as an addition reactant and an unreacted alcohol compound. Therefore, an aliphatic glycoside compound can be obtained by conveniently removing the alcohol compound by an ordinarily used method. Incidentally, since the aliphatic glycoside compound may be present in the inner part or on the surface of the cation exchanger, an increase in the yield of the aliphatic glycoside compound can be expected by collecting these aliphatic glycoside compounds from the cation exchanger by an ordinarily used method (for example, the above-described regeneration treatment).

As a result, it can be seen that the present invention can produce a high-purity aliphatic glycoside compound in which coloration as well as the contamination of by-products are highly suppressed.

Moreover, the production process of the present invention can accomplish the addition reaction by a convenient operation of bringing reactants into contact with an acid catalyst (preferably, a solid acid catalyst), under relatively mild conditions (atmospheric pressure, 60° C.) in Examples 1 to 4. Further, in a suitable embodiment of the present invention, the above-described separation step for the acid catalyst, and isolation and purification (purification step) of the aliphatic glycoside compound from the reaction mixture can also be conveniently carried out. Such a convenient production process can also be suitably applied to (established in), for example, a continuous method of passing through a column packed with a cation exchanger as a solid acid catalyst.

Moreover, since commercially available aliphatic glycoside compounds are relatively expensive, the present invention by which an aliphatic glycoside compound can be produced at a high conversion ratio (high purity) by a convenient process by using an intramolecularly dehydrated sugar, has high industrial applicability, even from the viewpoint of adding a new value of utilization to intramolecularly dehydrated sugars (thermal decomposition by-products of non-edible biomass).

Having described our invention as related to the present embodiments, it is our intention that the invention not be limited by any of the details of the description, unless otherwise specified, but rather be construed broadly within its spirit and scope as set out in the accompanying claims.

The present application claims priority of Japanese Patent Application No. 2020-094792 filed in Japan on May 29, 2020, which is herein incorporated by reference as part of the present specification.

1. A method of producing an aliphatic glycoside compound or a sugar fatty acid ester compound, which comprises a step of subjecting an intramolecularly dehydrated sugar and an alcohol or carboxylic acid compound of an aliphatic hydrocarbon to an addition reaction in the presence of an acid catalyst.

2. The method of producing an aliphatic glycoside compound or a sugar fatty acid ester compound according to claim 1, wherein the acid catalyst is a solid acid catalyst.

3. The method of producing an aliphatic glycoside compound or a sugar fatty acid ester compound according to claim 2, wherein the solid acid catalyst is a cation exchanger.

4. The method of producing an aliphatic glycoside compound or a sugar fatty acid ester compound according to claim 1, wherein the intramolecularly dehydrated sugar is an intramolecular dehydration reaction product in which water molecules are eliminated from two hydroxy groups including a hydroxy group bonded to a carbon atom at the 1-position in the cyclic structure.

5. The method of producing an aliphatic glycoside compound or a sugar fatty acid ester compound according to claim 1, wherein the intramolecularly dehydrated sugar is an intramolecularly dehydrated sugar of an aldose.

6. The method of producing an aliphatic glycoside compound or a sugar fatty acid ester compound according to claim 1, wherein the intramolecularly dehydrated sugar is levoglucosan.

7. The method of producing an aliphatic glycoside compound or a sugar fatty acid ester compound according to claim 1, wherein the number of carbon atoms constituting the alcohol or carboxylic acid compound is 1 to 22.

8. The method of producing an aliphatic glycoside compound or a sugar fatty acid ester compound according to claim 1, wherein the aliphatic hydrocarbon is a saturated aliphatic hydrocarbon.

9. The method of producing an aliphatic glycoside compound or a sugar fatty acid ester compound according to claim 1, wherein a mixture of the alcohol or carboxylic acid compound of an aliphatic hydrocarbon and the intramolecularly dehydrated sugar is brought into contact with the acid catalyst.

10. The method of producing an aliphatic glycoside compound or a sugar fatty acid ester compound according to claim 9, wherein the mixture is brought into contact with the acid catalyst by a batch method or a continuous method.

11. The method of producing an aliphatic glycoside compound or a sugar fatty acid ester compound according to claim 9, wherein the mixture is a mixed liquid obtained by dissolving at least a portion of the intramolecularly dehydrated sugar in the alcohol or carboxylic acid compound of an aliphatic hydrocarbon.
